(19) United States
(12) Patent Application Publication
(10) Pub. No.: US 2023/0364326 A1
(43) Pub. Date: Nov. 16, 2023

(54) IMPLANTABLE MEDICAL DEVICE AND SYSTEM

(71) Applicant: Cerebral Therapeutics, Inc., Aurora, CO (US)

(72) Inventors: Daniel J. Abrams, Denver, CO (US); Dwight Skinner, New Brighton, MN (US)

(21) Appl. No.: 18/196,506

(22) Filed: May 12, 2023

Related U.S. Application Data
(60) Provisional application No. 63/342,420, filed on May 16, 2022.

(51) Int. Cl. A61M 5/14; A61M 25/00

(52) U.S. Cl. CPC A61M 5/14; A61M 25/0026

(57) ABSTRACT

An implantable device includes a housing having a bottom configured to face the skull and a top configured to face a scalp when implanted. The device includes a first catheter connector configured to couple to a first dual lumen catheter. The first connector extends from the housing. The device comprises a second catheter connector configured to couple to a single lumen catheter. The second catheter connector extends from the housing. The device includes an opening defined by the top of the housing. The opening is configured to be accessed by a needle percutaneously inserted through the scalp when the device is implanted. The device includes a first fluid pathway from the first catheter connector to the opening defined by the top of the housing, and the device includes a second fluid pathway from the first catheter connector to the second catheter connector.

### CROSS REFERENCE TO RELATED APPLICATION

This application claims the benefit of priority to U.S. Provisional Patent Application No. 63/342,420, filed on 16 May 2022, the disclosure of which is incorporated herein by reference in its entirety.

### FIELD

The present disclosure relates to, among other things, implantable medical devices and systems for treating neurological disorders. The devices and systems may be used to deliver a fluid therapeutic composition to a brain of a subject, such as to a cerebrospinal fluid (CSF) containing compartment of the brain, withdrawing or draining fluid from a CSF-containing compartment, such as a lateral cerebral ventricle, or delivering fluid to the brain and withdrawing or draining fluid from the brain.

### INTRODUCTION

Delivery of therapeutic agents to the central nervous system (CNS) and treatment of diseases of the CNS present challenges. For example, many therapeutic agents are not able to reach the brain at therapeutic concentrations when administered through traditional routes due to the blood-brain-barrier (BBB). In addition, systemic concentrations of therapeutic agents, or metabolites or degradation products thereof, may be undesirably high to achieve therapeutic levels in the brain when therapeutic agents that do cross the BBB are systemically administered.

Some devices and therapies have been developed to administer therapeutic agents to CSF to address some of these challenges. Such devices and therapies have typically been configured to deliver a bolus of therapeutic agent to a cerebral ventricle or to chronically administer the therapeutic agent to intrathecal space of the spinal canal. Such approaches have shortcomings for treatment of diseases of the brain. For example, such approaches lack the ability to achieve adequate spatially and temporally exposure of a therapeutic agent. Bolus administration of a therapeutic agent may not provide consistent therapeutically effective concentrations of the therapeutic agent, and intrathecal administration may not provide for sufficiently high concentrations of therapeutic agent in the brain due to, for example, gravitational forces and relatively limited CSF circulation.

Additionally, such devices and therapies may introduce some risk to the patient. For example, repeated penetrations of the brain may increase the risk of a brain hemorrhage. As another example, devices implanted under the skin of a patient may be susceptible to puncture during sampling that can lead to infection or device malfunction. Other considerations for devices and therapies for the CNS may include, but are not limited to, cosmetic concerns, reoperation, patient comfort, and risk of infection.

Recently, implantable medical devices having separate infusion and fluid aspiration pathways have been developed. The devices include an access port for sampling CSF, via an attached catheter, through the aspiration pathway. The infusion pathway may be connected to an implantable infusion device to deliver fluid to the CSF via an attached catheter. The device is aligned with a burr hole created in the skull to allow introduction of the catheter or catheters into the brain. A portion of the device may be implanted in the burr hole, which may provide positional stability to the device.

Because the device is aligned with the burr hole, the device tends to be placed beneath scar tissue resulting from surgery associated with creating the burr hole and implanting the device. As such, insertion of the needle into the access portion to sample CSF via aspiration results in insertion of the needle through scar tissue. Sampling through scar tissue may reopen incisions or be more susceptible to infection because scar tissue can be avascular (e.g., lack blood vessels) and can be harder than surrounding tissue making it more difficult to penetrate into the sampling device.

### SUMMARY

The present application relates to, among other things, an implantable device having a first pathway for infusing fluid into a brain of a subject and a second pathway for withdrawing fluid from the brain of the subject, in which the device is configured to be implanted between the scalp and the skull of the subject and is configured to be offset from a burr hole through the skull. By having the device offset from the burr hole, an aspiration port of the device may be accessed by a needle inserted percutaneously through scalp tissue that is not scarred from surgery associated with creating the burr hole. When implanted, the device may be inserted through an opening in the scalp used to gain access to the skull for creating the burr hole.

The devices described herein may be, for example, advantageously employed in pediatric subjects in which the skull has not completely developed, relative to, for example, devices that are at least partially implanted in the burr hole. Skulls of pediatric subjects are typically thinner and the bone is more soft than skulls of adult subjects. Accordingly, the skulls of pediatric subjects will grow and thicken over time, which does not occur with adult skull bones. If a long-term or permanently implanted device having a snug fit is place in a burr hole of a pediatric subject, green stick fracturing and buckling of the bone at the edge of the burr hole may occur, which may result in bleeding, damage to the brain, or predisposing the drug infusion and CSF sampling channels to changing brain position, which may result in bleeding and damage to the brain in addition to potential loss of ability to infuse drug and sample CSF. Furthermore, the growth of bone around a device snugly implanted in a burr hole of a pediatric subject may make it harder to sample or make removal of the device more difficult or dangerous if revision, removal, or reposition is needed.

In an aspect described herein, a device is configured to be implanted between a scalp and a skull of a subject. The device comprises a housing having a bottom configured to face the skull, a top opposing the bottom, and a side connecting the top to the bottom. The device includes a first catheter connection port configured to couple to a first dual lumen catheter. The first catheter connection port extends from the side of the housing. The device comprises a second catheter connection port configured to couple to a single lumen catheter. The second catheter connection port extends from the housing. The device comprises an opening defined by the top of the housing. The opening is configured to be accessed by a needle percutaneously inserted through the scalp when the device is implanted. The device includes a first fluid pathway from the first catheter connection port to the opening, and the device includes a second fluid pathway from the first catheter connection port to the second catheter connection port.

A system may comprise the device and the dual lumen catheter. The system may also include the single lumen catheter. The system may also comprise an implantable infusion device operatively couplable to the single lumen catheter.

In an aspect described herein, a system comprises an access port device configured to be implanted between a scalp and a skull of a subject. The access port device comprises an opening configured to be accessed by a needle percutaneously inserted through the scalp when the access port device is implanted, a catheter connector, and a fluid pathway extending from the catheter connector to the opening. The system further comprises a first catheter, or a first portion of a single catheter, comprising a first lumen and a second lumen. The first catheter, or the first portion of the single catheter, comprises a first opening in communication with the first lumen and a second opening in communication with the second lumen. The first and second openings are configured to be implanted in a brain of the subject. The system further comprises a second catheter or a second portion of the single catheter. The second catheter is configured to be placed in communication with the first lumen and is configured to be connected to the catheter connector of the access port device. The second portion the single catheter comprises the first lumen and is configured to be connected to the catheter connector of the access port device. The system further comprises a third catheter or a third portion of the single catheter. The third catheter comprises a fourth lumen configured to be placed in communication with the second lumen and is configured to be connected to an implantable infusion pump. The third portion of the single catheter comprises the second lumen and is configured to be connected to the implantable infusion pump.

The system may further comprise a first catheter connector and/or a second catheter connector. The first catheter connector may be configured to operatively couple the second catheter to the first catheter or the first portion of the single catheter such that the third lumen is in fluid communication with the first lumen. The second catheter connector may be configured to operatively couple the third catheter to the first catheter or the first portion of the single catheter such that the fourth lumen is in fluid communication with the second lumen.

The system may further comprise the implantable infusion pump.

Also provided herein are methods of implanting the devices and systems described herein, as well as methods of treating, monitoring, or treating and monitoring a disease of a subject using the devices and systems described herein.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF THE DRAWINGS

FIG. 1 is a schematic side view of an embodiment of an implantable system illustrating some component devices.

FIG. 2 is a schematic top view of an embodiment of some components of the implantable medical system depicted in FIG. 1.

FIG. 3 is a schematic sectional view illustrating some components of an embodiment of an implantable device.

FIG. 4A is a schematic exploded side view of a catheter assembly.

FIG. 4B is a schematic front view of a catheter connector shown in FIG. 4A.

FIGS. 5A-C are schematic sectional views of the components of the assembly depicted in FIG. 4A, with FIG. 5A showing the distal catheter, FIG. 5B showing the catheter connector, and FIG. 5C showing the proximal catheter.

FIG. 6A is a schematic top view of an embodiment of an access port device.

FIG. 6B is a schematic bottom view of the access port device shown in FIG. 6A.

FIG. 7 is a schematic top view of an embodiment of some components of an implantable medical system.

FIG. 8 is a schematic side view of an embodiment of some components of the implantable system depicted in FIG. 7.

FIG. 9 is a schematic sectional view illustrating some components of an embodiment of an implantable device.

FIG. 10 is a schematic exploded side view of a catheter assembly.

FIG. 11 is a schematic front view of the catheter assembly shown in FIG. 10.

FIG. 12A is a schematic sectional view of the dual lumen catheter of the catheter assembly shown in FIG. 10.

FIGS. 12B-D are schematic sectional views of the connector of the catheter assembly shown in FIG. 10.

FIG. 12E is schematic sectional view of the first single lumen catheter of the catheter assembly shown in FIG. 10.

FIG. 12F is schematic sectional view of the second single lumen catheter of the catheter assembly shown in FIG. 10.

FIG. 13A is a schematic top view of an embodiment of an access port device.

FIG. 13B is a schematic bottom view of the access port device shown in FIG. 13A.

FIG. 14A is a schematic top view of an embodiment of an access port device.

FIG. 14B is a schematic bottom view of the access port device shown in FIG. 14A.

FIG. 15 is a schematic top view of an access port device implanted in a subject.

While the invention is susceptible to various modifications and alternative forms, specific embodiments thereof are shown by way of example in the drawings and may herein be described in detail. The drawings may not be to scale. It should be understood, however, that the drawings and detailed description thereto are not intended to limit the invention to the particular form disclosed, but on the contrary, the intention is to cover all modifications, equivalents and alternatives falling within the spirit and scope of the present invention as defined by the appended claims.

Like numbers used in the figures refer to like components. However, it will be understood that the use of a number to refer to a component in a given figure is not intended to limit the component in another figure labeled with the same number. In addition, the use of different numbers to refer to components in different figures is not intended to indicate that the different numbered components cannot be the same or similar to other numbered components

### DETAILED DESCRIPTION

The present disclosure relates to, among other things, implantable medical devices and systems for treating neurological disorders. The devices and systems may be used to deliver a fluid therapeutic composition to a brain of a subject, such to a CSF-containing compartment, such as cerebral ventricles, withdrawing or draining fluid from a CSF-containing compartment of the brain, such as a cerebral ventricle, or delivering fluid to the brain and withdrawing or draining fluid from the brain.

An access port device of the present disclosure is configured to be implanted between a scalp and a skull of a subject. The access port device is configured to operatively connect with a catheter that has a distal portion positioned in the brain, preferably in a CSF-containing compartment of the brain. The distal portion of the catheter comprises an opening through which fluid, such as CSF, may flow. The access port device comprises an opening in fluid communication with a lumen of the catheter when the catheter is operatively coupled with the device. A needle may be inserted into the opening of the access port device to place the needle in fluid communication with the lumen of the catheter. Fluid may be aspirated from the brain using the needle. The fluid may flow through the opening in the catheter, the lumen of the catheter, and into the needle.

The distal portion of the catheter is inserted into the brain through a burr hole in the skull. An incision may be made in the scalp to gain access to the skull to create the burr hole. The catheter may be inserted through the incision and through the burr hole to implant the distal portion of the catheter in the brain. The catheter may be coupled to the access port prior or after the port is implanted between the skull and the scalp. The access port may be inserted through the incision in the scalp and positioned adjacent, but offset from, the burr hole. Accordingly, when a needle is inserted through the scalp into the opening of the access port device to aspirate fluid from the brain, via the attached catheter, the needle may be inserted through tissue that has not been scarred resulting from the surgical incision.

Systems described herein may also include an implantable infusion pump. In embodiments, the implantable infusion pump is coupled to the access port device through a catheter. The access port device may comprise an infusion pathway that allows fluid infused through the catheter to exit the access port device and enter another catheter to deliver the infused fluid into the brain. In embodiments, the implantable infusion pump is not operatively coupled to the access port device and bypasses the access port device to deliver fluid to the brain of the subject.

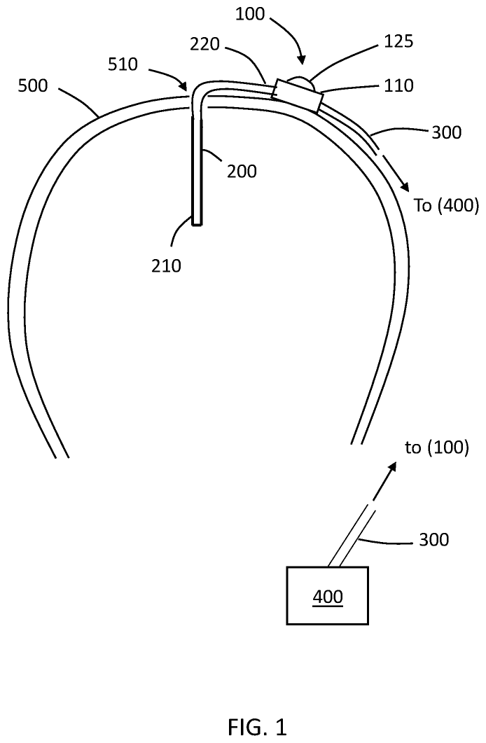

FIGS. 1-3 illustrate an embodiment of an access port device 100 and a system. The system comprises a catheter 200 or catheters having a distal portion 210 or portions having one or more openings (not shown in FIGS. 1-3) through which fluid may flow. Preferably, the distal portion 210 of the catheter 200 or catheters is positioned in the subject's CSF such that fluid may be delivered to or withdrawn from the subject's CSF through lumens of the catheter 200 or catheters. In embodiments, one or more lumens of the catheter 200 or catheters are placed in fluid communication with a cerebral ventricle of a subject such that fluid may be delivered to or withdrawn from the cerebral ventricle. In embodiments, the catheter 200 or catheters are configured such that one lumen may deliver fluid to brain tissue other than a cerebral ventricle and another lumen may deliver fluid to or withdraw fluid from a cerebral ventricle.

The distal portion 210 of the catheter 200 is implanted in the brain of the subject. A portion of the catheter 200 is received in a burr hole 510 in a skull 500 of the subject. A proximal end 220 of the catheter 200 is coupled to the access port device 100 implanted between the skull 500 and the scalp of the subject. The catheter 200 may be a dual lumen catheter. Alternatively, two catheters, each with a single lumen, may be employed. One lumen or catheter may be used to aspirate fluid from the brain of the subject. The other lumen or catheter may be used to infuse therapeutic fluid to the brain of the subject.

The proximal end 220 of the catheter 200 may be connected to the access port device 100 via a dual lumen catheter connector 135. In embodiments, the proximal end 220 of the catheter 200 is slid over the connector 135 to couple the catheter 200 to the device 100. The connector 135 may include barbs or other features, such as a compression fitting or the like, to retain the connected catheter 200. The connector 135 may include a slot 136 configured to receive a portion of the catheter 200 that separates one lumen from another in a dual lumen catheter. In embodiments, the connector 135 is configured to interact with the catheter 200 via interference fit.

The catheter connector 135 includes a first lumen in communication with an aspiration pathway 130 of the access port device 100 and a second lumen in communication with an infusion pathway 140 of the device 100. The infusion pathway 140 is in fluid communication with lumen of a second catheter connector 145, which is configured to connect to a single lumen catheter 300, which may be operatively coupled to an implantable infusion device 400. The infusion device 400 may infuse therapeutic fluid through the lumen of the catheter 300, through the infusion pathway 140, through a lumen of catheter 200, through an opening in the distal end 210 of the catheter 200, and into the brain of the subject. The infusion device 400 may be implanted at any suitable location in the subject. In embodiments, the infusion device 400 is implanted subcutaneously in a torso of the subject.

The proximal end 320 of the catheter 300 is connected to the access port device 100 via a connector 145. In embodiments, the proximal end 320 of the catheter 300 is slid over the connector 145 to couple the catheter 300 to the device 100. The connector 145 may include barbs of other features, such as a compression fitting or the like, to retain the connected catheter 300. In embodiments, the connector 145 is configured to interact with the catheter 300 via interference fit.

The access port device 100 comprises a housing 110 that defines an opening 127 through which an aspiration needle may be inserted. The device 100 may comprise a guide 150 to facilitate placement of the aspiration needle in communication with the aspiration pathway 130. The guide 150 may have any suitable shape. For example, the guide 150 or a portion thereof may have a conical shape.

The access port device 100 has a bottom surface 101 configured to be placed on a skull 500 of the subject when implanted. The device 100 has an opposing top surface 102 configured to face the scalp of the subject when implanted. Accordingly, an aspiration needle may be inserted through the scalp into the opening 127 defined by the top surface 102 of the device 100. A self-sealing septum 125 may be disposed across the opening 127.

The access port device 100 may comprise one or more through opening 160 that extends through the housing 110 and is configured to receive a fastener for retaining the device 100 to the skull. For example, the through opening 160 may be configured to receive a screw to secure the device 100 relative to the skull. In addition or alternatively, medical adhesive may be used to secure the bottom 101 of the access port device 100 to the skull.

The device 100 and system components may be implanted in any suitable manner. In embodiments, an incision is created in the scalp of the subject to provide access to the skull. A burr hole may then be generated in the skull. The distal portion 210 of the catheter 200 may be advanced through the incision and the burr hole to be implanted in the brain; e.g., in a cerebral ventricle. The proximal end 220 of the catheter 200 may be connected to the access port device 100 via the dual lumen catheter connector 135. The access port device 100 may be inserted through the incision and placed in proximity the burr hole 510 such that the bottom surface 101 of the device 100 rests on the skull 500. An intervening structure (not shown) may be placed between the bottom surface 101 of the device 100 and the skull 500. In either case, the bottom surface 101 of the device 100 faces the skull 500. The device 100 may be secured relative to the skull through the use of a fastener, such as a screw, inserted through through-hole 160. The through hole 160 may be located in proximity to the dual lumen catheter connector 135 of the device 100 so that the through hole 160 is located such that it can be accessed through the incision in the scalp.

The access port device 100 may be placed any suitable distance from the burr hole 510. In embodiments, a distance from the closest portion of the access port device 200 to the burr hole 510 is from about 0 centimeters to about 5 centimeters, such as from about 1 centimeter to about 3 centimeters.

The single lumen catheter 300 may be coupled to the access port device 100 via connector 145 before or after the device 100 is implanted. A distal portion of the single lumen catheter 300 may be tunneled to the location of the implantable infusion device 400 using any suitable technique, such as those know in the art.

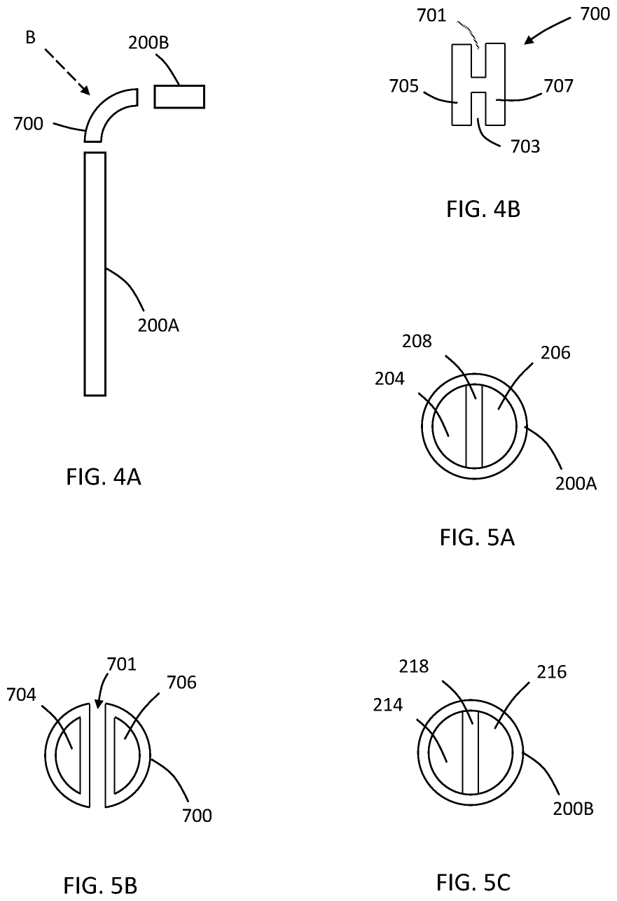

Referring now to FIG. 4-5, the system may include a first dual lumen catheter 200A (or two single lumen catheters), a second dual lumen catheter 200B (or two single lumen catheters), and a catheter connector 700 (or two catheter connectors). The first dual lumen catheter 200A may extend from a location within the brain (e.g., a cerebral ventricle) to about the top of the burr hole. The connector 700 may be used to connect the first dual lumen catheter 200A to the second dual lumen catheter 200B such that an aspiration lumen 204 of the first dual lumen catheter 200A is placed in fluid communication with an aspiration lumen 214 of the second dual lumen catheter 200B and such that an infusion lumen 206 of the first dual lumen catheter 200A is placed in fluid communication with an infusion lumen 216 of the second dual lumen catheter 200B. The catheter connector 700 may form a part of the first dual lumen catheter 200A or the second dual lumen catheter 200B rather than being a separate component. The connector 700 comprises a first lumen 704 to fluidly coupled the aspiration lumen 204 of the first dual lumen catheter 200A and the aspiration lumen 214 of the second dual lumen catheter 200B. The connector 700 comprises a second lumen 706 to fluidly coupled the infusion lumen 206 of the first dual lumen catheter 200A and the infusion lumen 216 of the second dual lumen catheter 200B.

The connector 700 may have any suitable size in shape. The connector 700 may be substantially straight or may be curved (e.g., as depicted in FIG. 4A). The connector 700 may comprises a 45-degree bend, a 90-degree bend, or the like.

In embodiments, the connector 700 has a first leg 705, through which lumen 704 runs, configured to be received in a lumen 204 of the first catheter 200A on one end and in a lumen 214 of the second catheter 200B on the other end. The connector 700 may also have a second leg 707, through which lumen 706 runs, configured to be received in a lumen 206 of the first catheter 200A on one end and in a lumen 216 of the second catheter 200B on the other end. The connector 700 has a slot 701 configured to receive a central body portion 218 of the second catheter 200B and has a slot 703 configured to receive a central body portion 208 of the first catheter 200A. The size, shape, configuration, etc. of the connector may change depending on the nature of the catheters employed.

The second dual lumen catheter 200B (or two single lumen catheters) may couple to the connector 700 and to the dual lumen catheter connector 135 of the access port device 100. The second dual lumen catheter 200B may be formed of material that may prevent accidental puncture if the aspiration needle misses the opening of the access port device when the needle is inserted through the scalp of the subject. The second dual lumen catheter 200 may be formed of puncture resistant hard plastic material or metallic material or may comprise a puncture resistant reinforcing element, such as a braid or mesh.

The second dual lumen catheter 200B may have any suitable length. In embodiments, the second dual lumen catheter 200B has a length from about 0.5 centimeters to about 5 centimeters.

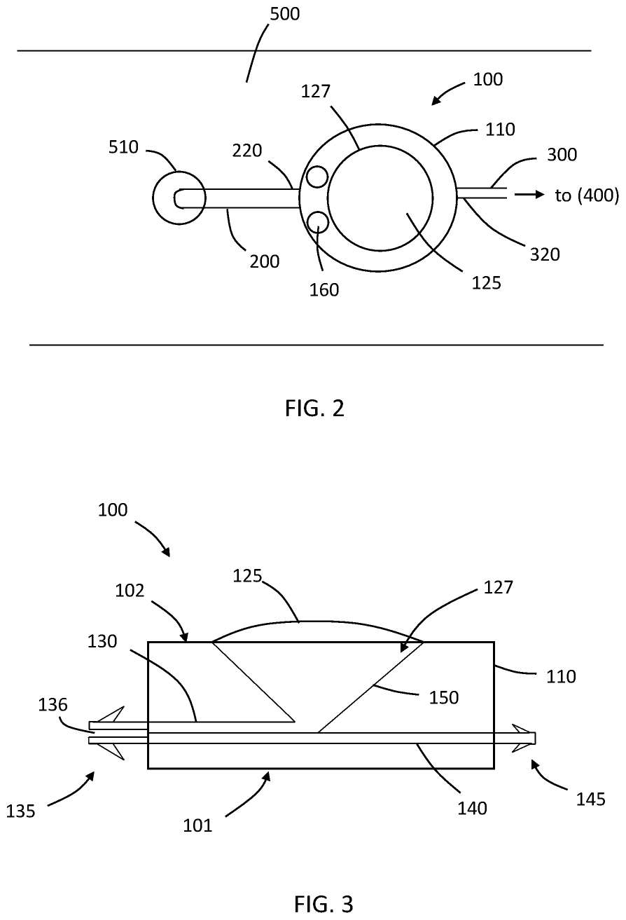

In embodiments, the system comprises a single dual lumen catheter (e.g., catheter 200 depicted in FIGS. 1-2) that extends into the brain (e.g., a cerebral ventricle) and couples to the access port device. The proximal portion, for example, the proximal most 0.5 centimeters to 5 centimeters, may be puncture resistant. For example, the proximal portion may be formed of puncture resistant hard plastic material or metallic material or may comprise a puncture resistant reinforcing element, such as a braid or mesh.

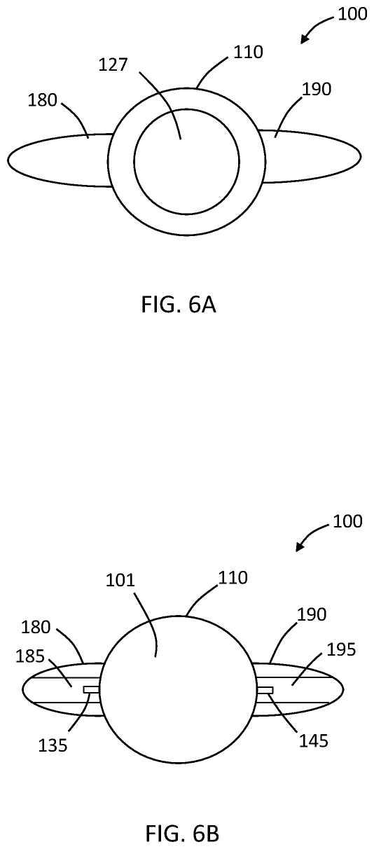

Referring now to FIGS. 6A-B, an access port device 100 having protective coverings 180, 190 is shown. The protective coverings 180, 190 extend from, or are a part of, the housing 110 of the device 100. The protective coverings 180, 190 are configured to protect the proximal portion/catheter 220 and the proximal portion 320 of catheter 300 from being accidentally being punctured by an aspiration needle if a percutaneously inserted needle misses the opening 127. If the protective coverings 180, 190 are separate from the housing 110 the coverings may be coupled to the housing in any suitable manner such as snap fit, or the like. The protective coverings 180, 190 may be formed from any suitable material, such as a hard plastic material, a glass material, a ceramic material, a metallic material, or the like or combinations thereof.

The protective coverings 180, 190 may extend from a side of the device 100 any suitable distance. In embodiments, protective covering 180 extends to at least partially cover the burr hole. In embodiments, the protective coverings 180, 190 extend from the side of the device 100 about 0.5 centimeters to about 5 centimeters.

The bottom of the protective coverings 180, 190 may comprises grooves 185, 195 configured to receive distal portion/catheter 220 and catheter 300, respectively. The protective coverings 180, 190 are configured and arranged to permit coupling of distal portion/catheter 220 to catheter connector 135 and to permit coupling of catheter 300 to catheter connector 145.

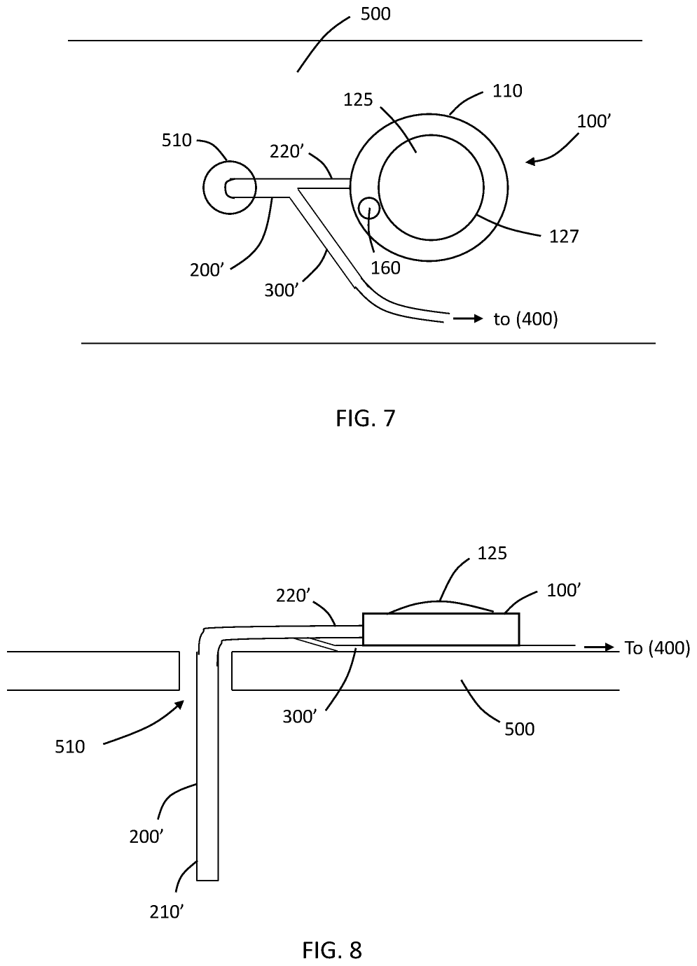

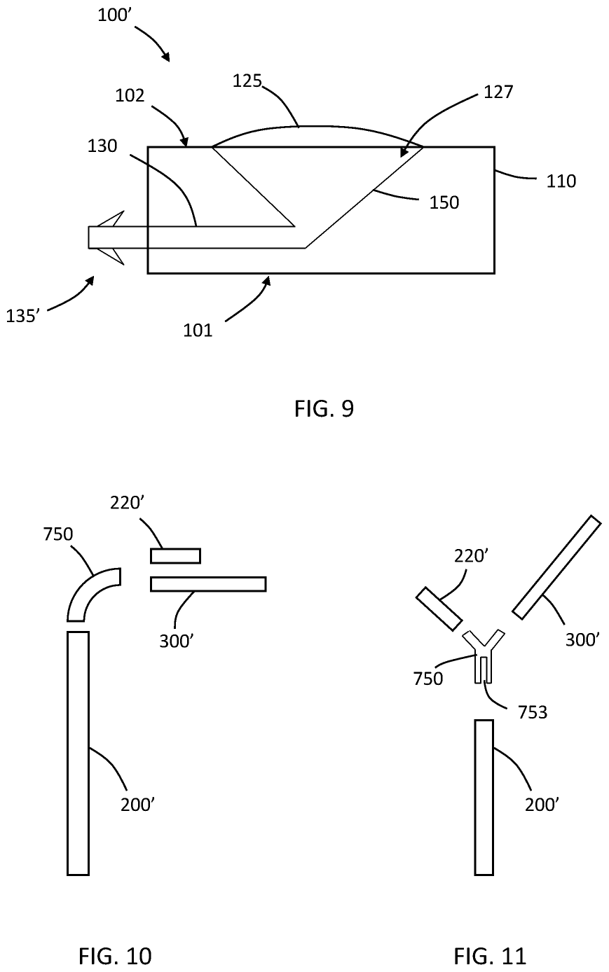

Referring now to FIGS. 7-9, embodiments of an access port device 100 and systems are shown. The access port device 100′ in FIGS. 7-9 differs from the access port device 100 in FIGS. 1-3 in that the access port device 100′ in FIGS. 7-9 does not include an infusion pathway. Rather, the infusion pathway runs through a catheter 300′ that is separate from the device 100′. The catheter 300′ may be a bifurcated leg, constituting a single lumen portion, of a dual lumen catheter 200′ or may be operatively coupled to a lumen of the dual lumen catheter 200′ through a connector.

The system depicted in FIGS. 7-9 comprise a catheter 200′ having a distal portion 210′ comprising one or more openings through which fluid may flow. Preferably, the distal portion 210′ of the catheter 200′ is positioned in the subject's CSF such that fluid may be delivered to or withdrawn from the subject's CSF through lumens of the dual catheter 200′. In embodiments, one or both lumens of the dual lumen catheter 200′ are placed in fluid communication with a cerebral ventricle of a subject such that fluid may be delivered to or withdrawn from the cerebral ventricle. In embodiments, the catheter 200′ or catheters are configured such that one lumen may deliver fluid to brain tissue other than a cerebral ventricle and another lumen may deliver fluid to or withdraw fluid from a cerebral ventricle. The distal portion 210′ of the catheter 200′ is implanted in the brain of the subject. A portion of the catheter 200′ is received in a burr hole 510 in a skull 500 of the subject.

Catheter 220′ may be a proximal end portion, single lumen extension of the dual lumen catheter 200′ or may be operatively coupled to a lumen of the dual lumen catheter 200′ through a connector. The proximal end or proximal catheter 220′ is coupled to the access port device 100′ implanted between the skull 500 and the scalp of the subject. Catheter 200′ is a dual lumen catheter with one lumen (the aspiration lumen) operatively coupled to the access port device 100′ and with the other lumen (the infusion lumen) operatively coupled to an implantable infusion device (e.g., device 400 depicted in, and discussed above regarding, FIG. 1).

The proximal end/catheter 220′ may be connected to the access port device 100′ via a catheter connector 135′. In embodiments, the proximal end/catheter 220′ is slid over the connector 135′ to couple the proximal end/catheter 220′ to the device 100′. The connector 135′ may include barbs or other features, such as a compression fitting or the like, to retain the connected proximal end/catheter 220′. In embodiments, the connector 135′ is configured to interact with the proximal end/catheter 220′ via interference fit. The catheter connector 135′ includes a lumen in communication with an aspiration pathway 130 of the access port device 100′.

The access port device 100′ comprises a housing 110 that defines an opening 127 through which an aspiration needle may be inserted. The device 100 may comprise a guide 150 to facilitate placement of the aspiration needle in communication with the aspiration pathway 130. The guide 150 may have any suitable shape. For example, the guide 150 or a portion thereof may have a conical shape.

The access port device 100′ has a bottom surface 101 configured to be placed on a skull 500 of the subject when implanted. The device 100′ has an opposing top surface 102 configured to face the scalp of the subject when implanted. Accordingly, an aspiration needle may be inserted through the scalp into the opening 127 defined by the top surface 102 of the device 100′. A self-sealing septum 125 may be disposed across the opening 127.

The access port device 100′ may comprise a through opening 160 that extends through the housing 110 and is configured to receive a fastener for retaining the device 100′ to the skull. For example, the through opening 160 may be configured to receive a screw to secure the device 100′ relative to the skull. In addition or alternatively, medical adhesive may be used to secure the bottom 101 of the access port device 100′ to the skull 500.

The device 100′ and system components may be implanted in any suitable manner. In embodiments, an incision is created in the scalp of the subject to provide access to the skull 500. A burr hole 510 may then be generated in the skull. The distal portion 210′ of the dual lumen catheter 200′ may be advanced through the incision and the burr hole to be implanted in the brain; e.g., in a cerebral ventricle. The proximal end/catheter 220′ may be connected to the access port device 100′ via the dual lumen catheter connector 135′. The access port device 100′ may be inserted through the incision and placed in proximity the burr hole 510 such that the bottom surface 101 of the device 100′ faces the skull 500. In embodiments, the bottom surface 101 of the device 100 rests on the skull 500. The device 100 may be secured relative to the skull through the use of a fastener, such as a screw, inserted through through-hole 160. The through hole 160 may be located in proximity to the dual lumen catheter connector 135 of the device 100 so that the through hole 160 is located such that it can be accessed through the incision in the scalp.

The access port device 100′ may be placed any suitable distance from the burr hole 510. In embodiments, a distance from the closest portion of the access port device 200 to the burr hole 510 is from about 0 centimeters to about 5 centimeters, such as from about 1 centimeter to about 3 centimeters.

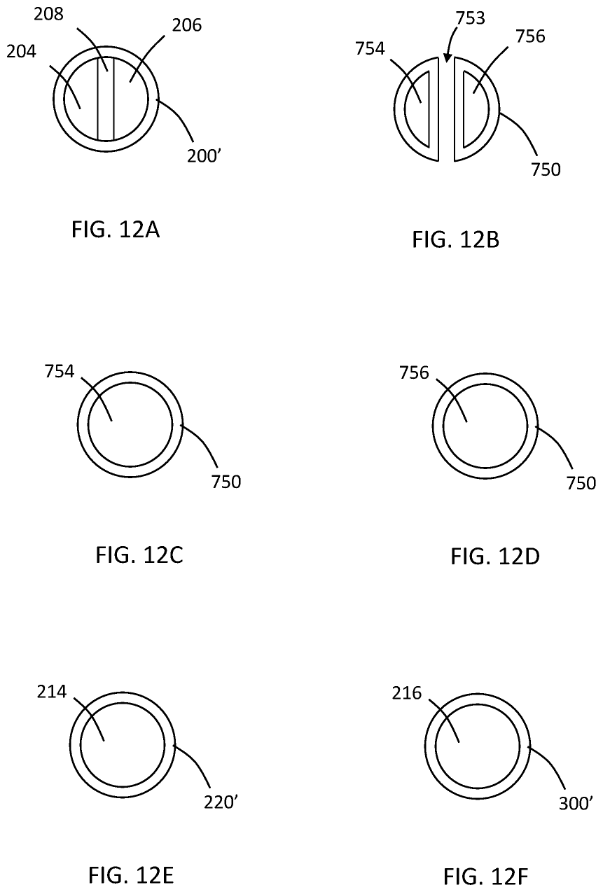

Referring now to FIG. 10-12, the system of FIGS. 7-9 may include a first dual lumen catheter 200′, a first single lumen catheter 220′, a second single lumen catheter 300′, and one or more catheter connectors 750. The dual lumen catheter 200′ may extend from a location within the brain (e.g., a cerebral ventricle) to about the top of the burr hole. The connector 750 may be used to connect the dual lumen catheter 200′ to the first single lumen catheter 220′ such that an aspiration lumen 204 of the dual lumen catheter 200′ is placed in fluid communication with the lumen 214 of catheter 220′ and may be used to connect the dual lumen catheter 200′ to the second single lumen catheter 300′ such that an infusion lumen 206 of the dual lumen catheter 200′ is placed in fluid communication with the 216 lumen of catheter 300′. Alternatively, two individual connectors may be used to separately connect the first 220′ and second 300′ single lumen catheters to the dual lumen catheter 200′.

The catheter connector 750 may form a part of the dual lumen catheter 200′ rather than being a separate component.

The connector 750 comprises a first lumen 754 to fluidly coupled the aspiration lumen 204 of the dual lumen catheter 200′ and the lumen 214 of the catheter 220′. The connector 750 comprises a second lumen 756 to fluidly coupled the infusion lumen 206 of the dual lumen catheter 200′ and the lumen 216 of catheter 300′.

The connector 750 may have any suitable size in shape. The connector 750 may be substantially straight or may be curved (e.g., as depicted in FIG. 10). The connector 750 may comprises a 45-degree bend, a 90-degree bend, or the like.

In embodiments, the connector has a first leg (through which lumen 754 runs) configured to be received in a lumen 204 of the dual lumen catheter 200′. The connector 700 may also have a second leg (through which lumen 706 runs) configured to be received in a lumen 206 of the dual lumen catheter 200′. The connector 750 has a slot 753 configured to receive a central body portion 208 of the dual lumen catheter 200′. The connector 750 has a first arm (through which lumen 754 runs) configured to be received in the lumen 214 of catheter 220′. The connector 750 has a second arm (through which lumen 756 runs) configured to be received in the lumen 216 of catheter 300′.

The size, shape, configuration, etc. of the connector may change depending on the nature of the catheters employed. In the embodiment depicted in FIG. 11, the connector 750 has a “Y” shape. Of course, the connector may have any other suitable shape.

Catheter 220′ may couple to the connector 750 and to the catheter connector 135′ of the access port device 100′. Catheter 220′ may be formed of material that may prevent accidental puncture if the aspiration needle misses the opening of the access port device when the needle is inserted through the scalp of the subject. Catheter 220′ may be formed of puncture resistant hard plastic material or metallic material or may comprise a puncture resistant reinforcing element, such as a braid or mesh.

Catheter 200′ may have any suitable length. In embodiments, catheter 200′ has a length from about 0.5 centimeters to about 5 centimeters.

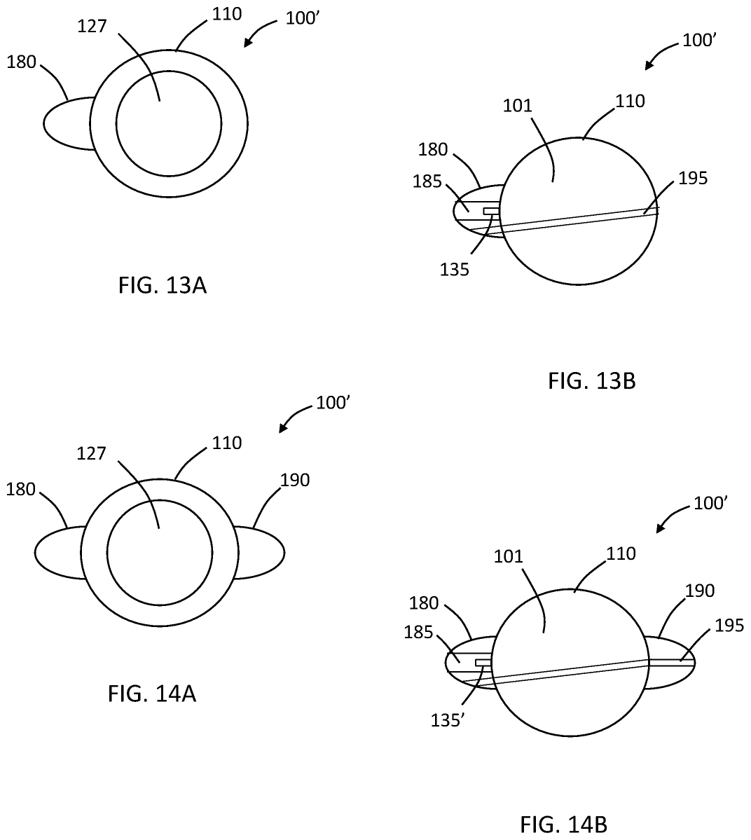

Referring now to FIGS. 13-14, access port devices 100′ having protective covering 180 or coverings 180, 190 are shown. The protective coverings 180, 190 extend from, or are a part of, the housing 110 of the device 100′. The protective covering 180 is configured to protect the catheter 220 and may be configured to protect catheter 300′ from being accidentally being punctured by an aspiration needle if a percutaneously inserted needle misses the opening 127. Protective covering 190, if present, is configured to protect catheter 300′. If the protective coverings 180, 190 are separate from the housing 110 the coverings may be coupled to the housing in any suitable manner such as snap fit, or the like. The protective coverings 180, 190 may be formed from any suitable material, such as a hard plastic material, a glass material, a ceramic material, a metallic material, or the like or combinations thereof.

The protective coverings 180, 190 may extend from a side of the device 100 any suitable distance. In embodiments, protective covering 180 extends to at least partially cover the burr hole. In embodiments, the protective coverings 180, 190 extend from the side of the device 100 about 0.5 centimeters to about 5 centimeters.

The bottom of the protective coverings 180, 190 may comprises grooves 185, 195 configured to receive catheter 220′ and catheter 300′, respectively. Protective coverings 180 is configured and arranged to permit coupling of catheter 220′ to catheter connector 135′. Groove 195 may extend across the bottom 101 of the housing 110 of the device 100′ and may extend across a portion of protective covering 180.

Groove 195 may receive catheter 300′ when the bottom 100 of the device 100′ rests on the skull 500 of the subject such that the bottom 101 may rest on the skull 500 without pinching or deforming the catheter 300′. Alternatively, catheter 300 may be directed away from the device 100′ so that the likelihood of accidental penetration by an aspiration needle is minimized.

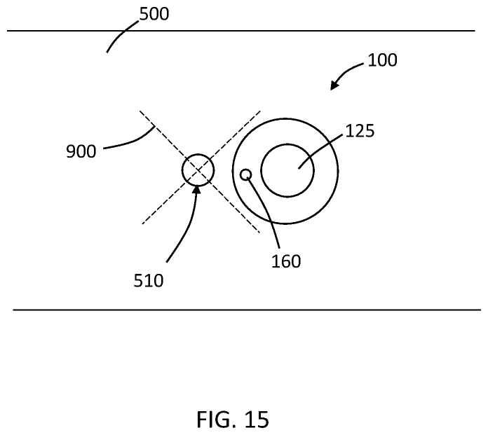

Referring now to FIG. 15, a schematic diagram illustrating an access port device 100 (e.g., device 100 or 100′ depicted in the preceding figures) implanted relative to a burr hole 510 of the skull 500 of a subject is shown. Also shown in dashed lines is the relative location of a scar tissue 900 resulting from an incision in the scalp during the implantation process. During implantation, the burr hole 510 may be created and the device 100 may be inserted through the scalp at the incision and placed in proximity to the burr hole 510. A screw or other fastener may be inserted through through-hole 160 to secure the device 100 relative to the burr hole 510. As shown, the septum 125 is positioned at a location away from the scar tissue 900, allowing an aspiration needle to be inserted through the scalp through tissue that is not scarred to access the septum to aspirate fluids from the brain of the subject as described above.

The access port devices described herein may be formed from any suitable material. In embodiments, the housing of the access port device is formed from a hard plastic material, a glass material, a ceramic material, a metal material, or the like, or a combination thereof. Similar materials may be used to form the fluid pathways of the access port devices, catheter connectors, and penetration resistant catheters or portions thereof. Some examples of materials include high performance thermoplastics or relatively rigid plastic materials, such as polyurethane, polycarbonate, polysulfone, polyether ether ketone (PEEK), nylon, and Ultra High Molecular Weight Polyethylene (UHMWPE); and a biocompatible metal, such as a stainless-steel alloy, titanium, and nitinol. Preferably, the material is compatible with magnetic resonance imaging (MRI). Preferably, the housing comprises a biocompatible material or comprises an exterior biocompatible coating.

The access port devices may have any suitable dimensions. Preferably, the height (bottom to top) of the device is sufficiently small to be well tolerated by a subject when implanted between the skull and the scalp of the subject. In embodiments, the height of the access port device is in a range from about 3 millimeters to about 8 millimeters. For example, the height of the access port device may be in a range from about 4 millimeters to about 6.5 millimeters or from about 5 millimeters to about 6.5 millimeters.

The access port devices may have any suitable shape. In embodiments, the access port devices have a generally circular cross-sectional shape. In embodiments, the access port devices have a diameter in a range from 15 millimeters to 30 centimeters. In embodiments, the access port devices have a generally polygonal cross-sectional shape. In embodiments, the access port devices have a generally elliptical cross-sectional shape. In embodiments, the access port devices have a cross-sectional shape that includes straight and/or curved edges.

The access port devices may have a convex shaped top surface. In embodiments, the thickness or height of the device increases moving from an outer edge of the device towards the center the device. The access port devices may have a concave shaped bottom surface to allow the devices to sit relatively flat on the skull.

The catheters described herein may have any suitable length and may be formed from any suitable material or materials. In embodiments, a catheter configured to extend from a burr hole to a cerebral ventricle may have a length from about 55 millimeters to about 80 millimeters, such as from about 60 millimeters to about 70 millimeters, or from about 62 millimeters to about 68 millimeters. Such lengths may be suitable for the catheter to extend from the burr hole to a lateral cerebral ventricle.

The catheters described herein may have any suitable outer diameter. For example, a dual lumen catheter may have an outer diameter is from about 2 mm to about 4 mm, such as from about 2 mm to about 3 mm, or from about 2 mm to about 2.5 mm.

The lumens in a dual lumen catheter may be oriented in any suitable manner. For example, the two lumens may be concentric, such as a lumen within a lumen (or catheter within a catheter) or may be side-by-side. In embodiments, a dual lumen catheter has two semicircular (or D-shaped) cross-sectional shaped lumens running along the length of the catheter. The semicircular lumens may be of any suitable size. For example, the inner dimension of the semicircular lumens at their largest width may be in a range from about 0.9 millimeters to about 1.5 millimeters, such as from about 1.1 millimeters to about 1.5 millimeters, or from about 1.2 millimeters to about 1.4 millimeters. The inner dimension of the semicircular lumens at their smallest width may be in a range from about 0.4 millimeters to about 0.7 millimeters, such as from about 0.5 millimeters to about 0.6 millimeters, or from about 0.55 millimeters to about 0.6 millimeters. The inner dimensions of the first and second lumens may be the same or different. Preferably, the inner diameter of the first and second lumen are the same or substantially the same (e.g., do not vary by more than 10%).

Preferably, the catheters described herein are biocompatible and compatible with a therapeutic fluid that may be delivered through a lumen of the catheter or CSF that may be withdrawn through a lumen of the catheter. Preferably, the material is biodurable. A biodurable material is a material that is compositionally and structurally stable for extended periods of time in a biological environment. Products made from such materials should not exhibit substantial breakdown, degradation, erosion, or deterioration of mechanical properties relevant to their employment when exposed to biological environments for periods of time commensurate with the use of the implantable device. An intended biological environment can be understood to be in vivo, i.e. associated with an implantable device in a patient. The period of implantation of a brain catheter described herein may be weeks, months, or years. For example, a catheter may be made from materials that are biodurable for at least 29 days, such as at least one year, at least three years, or at least five years.

Any suitable homopolymer, copolymer, blends of polymers or combinations of polymers may be used to form a catheter for insertion into the brain. Preferably, the polymers used to make the catheters are flexible during both fabrication and assembly. Preferably, the catheters are formed from materials that result in a flexible and soft catheter during its in vivo implantation period.

Preferably, the chemical composition and molecular structure is selected so that the catheter materials are flexible and soft but still remain biodurable and resist substantial breakdown, degradation, erosion, or deterioration of mechanical properties when exposed to therapeutic agents or excipients delivered over extended periods of time. Flexibility and softness are characteristics that tend to cause a catheter to lack biodurability and long-term compatibility in oxidative, hydrolytic, and body fluid contact environments. Accordingly, the choice of polymers should be carefully selected to achieve sufficient compatibility with the therapeutic agent and excipients, biodurability, flexibility, and softness.

Depending on compatibility with the therapeutic agent and excipients to be employed, the brain catheter may comprise cross-linked silicone, which may form flexible catheters. The glass transition temperature of cross-linked silicone may depend on the cross-link density. In some embodiments, the brain catheter may comprise cross-linked silicone having a Tg below −40° C., such as below −90° C. In some instances, components of therapeutic fluids may interact with a cross-linked silicone catheter. In some instances, components of therapeutic fluids may leach or extract components or parts of a cross-linked silicone catheter. Swelling and leaching or extraction may affect the biodurable nature of soft and flexible cross-linked silicone and its capability to maintain dimensions or wall thickness and structure to provide multiple stable fluid paths over extended periods of time.

In some embodiments, a brain catheter comprises polyurethane. Preferably, the brain catheter comprising polyurethane resists degradation in oxidative, hydrolytic, and body fluid contact environments. The polyurethane may comprise biostable hard and soft segments. The polyurethane may comprise a higher hard segment content. Such polyurethanes may resist degradation in oxidative, hydrolytic, and enzymatic changes under physiological conditions and body fluid contact. In some embodiments, the brain catheter comprises a polyurethane containing hard and soft segments that are formed by reacting a diol or polyol (an alcohol with more than two reactive hydroxyl groups per molecule) with a diisocyanate or a polymeric isocyanate in the presence of suitable catalysts and additives. The isocyanates (sometime chain extended with diols) form the hard segments and the polyols form the soft segments, with the segments linked by the urethane bonds formed from the reaction between the polyols and diisocyanate.

In some embodiments, the hard segment of the polyurethane comprises of an aromatic isocyanate. In some embodiment, the hard segment of the polyurethane comprises an aliphatic isocyanate. Preferably, the hard segment comprises an aromatic isocyanate as.

In some embodiments, the soft segment of the polyurethane comprises of a polyether. In some embodiments, the soft segment of the polyurethane comprises of a polycarbonate. The soft segment may provide suitable flexibility for use in single lumen catheters and multi-lumen catheters.

In some embodiments, a brain catheter comprises a polyurethane that is semi-crystalline with higher hardness. The semi-crystalline nature of the polyurethane polymers makes them resistant to swelling and leaching when in contact with CSF, therapeutic fluids, and therapeutic fluids having high concentration therapeutic agents. Semi-crystalline polyurethanes may be resistant to degradation in oxidative, hydrolytic, and enzymatic changes and body fluid contact environments. Higher hardness may provide a polyurethane resistant to swelling and leaching when in contact with CSF, therapeutic agents, and highly concentrated therapeutic agents. Higher hardness polyurethanes may be resistant to degradation in oxidative, hydrolytic, and enzymatic changes and body fluid contact environments. A polyurethane with a higher hardness (i.e. higher A or preferably D hardness scale) may have less soft segment and higher crystallinity, which may slow diffusion and interaction with solvents and solutions. Accordingly, brain catheters comprising polyurethane may be less susceptible to swelling or attack by solvents and solutions of therapeutic fluids.

A brain catheter comprising a polyurethane with higher hardness may allow for reduced wall thickness and may provide for processing advantages for single lumen brain catheters and multi-lumen brain catheters.

In some embodiments, the catheter may be formed from a material having a Shore A hardness in a range from about 70 A to about 110 A. In some embodiments, the catheter may be formed from a material having a Shore A hardness above 80 A or preferably above 90 A. In some embodiments, the catheter is formed from a material having a Shore D hardness of about 30D to about 70D, such as from about 40D to about 60D, or from about 50D to about 60D.

The brain catheter may comprise a semicrystalline polymers other than a polyurethane. The semicrystalline polymers may be selected to provide resistance to degradation in oxidative, hydrolytic, and enzymatic changes and body fluid contact environments while maintaining the desired flexibility through thinner wall or lower wall thickness. In some embodiments, a brain catheter comprises one or more of polyolefin, polyethylene, a fluorinated polymer, a fluorinated homopolymer, a fluorinated copolymer, a homopolymer of polyvinylidene difluoride (PVDF), a copolymer of PVDF, a copolymer of tetrafluoroethylene (TFE) and hexafluoro propylene (HFP), and polychlorotrifluoroethylene. A polyolefin may provide resistance to change during its in vivo life through its high crystallinity and long hydro-carbon chains, and a fluorinated polymer may provide resistance to change during its in vivo life through its high crystallinity and the inert nature of fluorine.

Some examples, of polymers that may be used to form a brain catheter as described herein include aliphatic or aromatic, polycarbonate-based thermoplastic polyurethane, such as CARBOTHANE (available from Lubrizol, Wickliffe, Ohio, USA), perfluoroelastomers, such as KALREZ (available from DuPont, Wilmington, Delaware, USA); PVDF, such as KYNAR FLEX (available from Daikin, Osaka, Japan) or KYNAR ULTRAFLEX (available from Arkema, Colombes, France); fluorinated ethylene propylenes, such as NEOFLON (available from Daikin, Osaka, Japan).

Exterior surfaces of the brain catheter preferably comprise material that are biocompatible. Preferably, exterior surfaces of the brain catheter inhibit tissue adhesion. Preferably, exterior surfaces of the brain catheter are easily inserted. A hydrophilic coating, such as a hydrogel, may be applied to an exterior surface to cause the exterior surface to be lubricious to facilitate insertion of the catheter into the brain. The exterior surface of the catheter may be coated with polytetrafluorethylene (PTFE) or another polymer to improve insertability, decrease adhesion, or increase insertability and decrease adhesion. In some embodiments, the structural material of the catheter is sufficiently insertable and sufficiently resists tissue adhesion without an additional coating. If a coating is applied, the surface of the catheter preferably includes functional groups that may covalently bind with a coating. The surface of the catheter may be treated to introduce functional groups, or the functional groups may be present in the material forming the catheter.

The brain catheter may include or may be coated with an antimicrobial material, such as antimicrobial silver or an antibiotic. In some embodiments the brain catheter includes or is coated with a composition comprising a combination of minocycline and rifampin. In some embodiments, the brain catheter is soaked in a solution comprising an antimicrobial agent, and the antimicrobial agent is taken up by the material forming the brain catheter.

The brain catheter may comprise radiopaque material visible by imaging, such as X-ray or magnetic resonance imaging (MRI). The brain catheter may comprise radiopaque material throughout the catheter, may comprise a concentrated area of radiopaque material, or may comprise radiopaque material throughout the catheter and may comprise a concentrated area of radiopaque material. The brain catheter may comprise a concentrated radiopaque material at the distal end portion. The brain catheter may comprise one or more concentrated radiopaque bands or markings along the length of the marker that may be used to determine the depth of the catheter during or after implantation.

The brain catheter may comprise any suitable radiopaque material. Examples of suitable radiopaque material includes barium sulfate, tantalum, and titanium.

In some embodiments, barium sulfate is blended with the polymer forming the catheter such that the barium sulfate is distributed through the catheter. Any suitable concentration of barium sulfate may be used. In some examples, about 5% barium sulfate by weight to about 20% barium sulfate by weight is blended into the polymer forming the catheter. For example, about 10% barium sulfate by weight to about 15% barium sulfate by weight, or about 12% barium sulfate may be blended into the polymer forming the catheter. 12% barium sulfate blended into the polymer was empirically determined to provide a suitable balance of a number of factors, including (i) ability to visualize the catheter throughout the implant process as it penetrates, (ii) manufacturability of thin-walled dual lumen catheter, and (iii) compatibility with fluids comprising high concentration therapeutic agents that may be infused through the catheter. Balancing these factors may achieve biocompatibility and biostability for the life of its implant, such as 5 years or more. In some embodiments, a tantalum marker, such as a tantalum bead, is positioned in the brain catheter at the distal end portion.

The brain catheter may comprise one or more openings in communication with a lumen of the catheter through which fluid may flow. The brain catheter may comprise any suitable number of openings in communication with each lumen. For example, the brain catheter may comprise one to ten or more openings in communication with each lumen, such as two to six openings or three to four openings in communication with each lumen. The brain catheter may have the same number of openings in communication with the first lumen as in communication with the second lumen. The brain catheter may have a different number of openings in communication with the first lumen than in communication with the second lumen.

The openings may be of any suitable size and may be configured in any suitable manner. The openings have a diameter or width of from about 0.2 millimeters to about 1 millimeter, such as from about 0.4 millimeters to about 0.6 millimeters, or about 0.5 millimeters. The openings may have the same or different diameters or widths.

The openings may be any suitable shape. For example, the openings may have circular or elliptical cross-sectional shapes, rectangular cross-sectional shape, triangular cross-sectional shape, or the like, or combinations thereof.

The openings may always be open or may be configured to open due to a pressure differential in the lumen of the catheter and CSF in which the distal end portion of the brain catheter is implanted. For example, the openings may comprise slits that open due to relative positive pressure in the lumen when fluid is infused through the lumen to the CSF or due to relative negative pressure in the lumen when CSF is aspirated through the lumen. The slit may be cut into a resilient material that flexes when under pressure but returns to an original shape as pressure equalizes.

The openings may be positioned at any suitable location of the catheter. In some embodiments, the one or more openings of the brain catheter in communication a lumen configured to infuse therapeutic fluid (e.g., the lumen in communication with the second fluid path of the implantable cranial medical device) may be positioned a short distance from the distal tip of the brain catheter. The one or more openings of the brain catheter in communication with a lumen through which CSF is configured to be aspirated (e.g., the lumen in communication with the first fluid path of the implantable cranial medical device) may be positioned at or in proximity to the distal tip. Separating infusion openings from aspiration openings may allow for infused fluid to mix with CSF so that aspirated CSF better represents concentrations of therapeutic agent in CSF than if the infusion and aspiration openings were in proximity to each other. If the infusion and aspiration openings are located adjacent to one another, then aspirated CSF may have higher concentration that CSF at a location more remote from the aspiration lumen. If the aspiration and infusion openings are located further apart from one another, then the aspirated fluid would be more representative of the entire CSF.

In addition to positioning aspiration and infusion openings longitudinally apart (closer to or further from the distal tip), the aspiration openings and the infusion openings may be placed on generally opposing sides of the brain catheter. For example, the aspiration openings and the infusion openings may be positioned from about 160 degrees to about 180 degrees radially apart from one another. By positioning the infusion ad aspiration lumens radially apart, substantial mixing of CSF with therapeutic fluid infused through an infusion opening may occur prior to aspirating the CSF through an aspiration opening.

In addition to positioning aspiration and infusion openings away from each other in a staggered manner in the cerebral ventricle, the position of the openings may be substantially separated such that the infusion holes are placed within a tissue location in the brain other than the cerebral ventricle, but the aspiration openings remain in the CSF.

The lumens of the brain catheter may have the same or different lengths. The lumens of the brain catheter may have a length that extends from a proximal end portion, which may be coupled to the brain catheter connector of the implantable cranial medical device, to the distal-most opening in the catheter in communication with the respective lumen. If the lumens of the brain catheter are configured to carry fluid to or from the same brain location, such as a cerebral ventricle, the lumens may have the same or substantially similar lengths. If the lumens of the brain catheter are configured to carry fluid to or from different brain locations, such as a ventricle and brain parenchyma, the lumens may be of substantially different lengths. For purposes of the present disclosure, lumens having “substantially different” lengths are lumens that have lengths that differ by more than 10 percent. Lumens that have “substantially similar” lengths are lumens that have lengths that differ by 10 percent or less.

In some embodiments, the brain catheter comprises multiple infusion openings positioned a distance from the distal tip, such as from about 3 millimeters from the distal tip to about 15 millimeters from the distal dip. For example, all the infusion openings may be positioned from about 3 millimeters to about 10 millimeters from the distal dip, or from about 4 millimeters to about 8 millimeters from the distal tip. In some embodiments, at least one aspiration lumen is positioned at the distal tip of the brain catheter.

A septum may be disposed across the opening of the access port devices described herein. Preferably, the septum is a self-sealing septum. A self-sealing septum may allow multiple cycles of needle insertion into and withdrawal from the port while continuing to seal the port from an interstitial environment in which the port may be located when implanted. The implantable cranial medical device may comprise any suitable self-sealing septum. For example, the self-sealing septum may comprise silicone, a polyethylene, of the like, and combinations thereof.

The port may be configured to receive a needle of any suitable gauge. The needle may be inserted through the septum to aspirate fluid from the subject through the aspiration pathway of the access port device and the aspiration lumen of the brain catheter. The port may comprises a guide, which may be conical shaped. The tapered nature of the guide (e.g., ferrule or funnel) may accommodate needles of a variety of sizes. For example, the guide (ferrule or funnel) may accommodate needles with a range of sizes from about 16 gauge to about 25 gauge, such as from about 18 gauge to about 22 gauge.

Preferably, access port devices described herein are configured to receive non-coring needles, such as Huber needles or butterfly needles. Non-coring needles may be designed with a deflected or offset ‘B’ bevel point. Such a tip has the advantage of parting rather than cutting a plug from or coring the septum of the port and may create a more comfortable injection. Using a non-coring needle, such as a Huber needle, may preserve the integrity of the septum and may prevent a plug of septum material from being cut and passed into the CSF.

In some embodiments, a kit may include one or more non-coring needles.

The implantable access port devices and associated devices described herein may be used in any suitable mater. If the distal end portion of the brain catheter is positioned in a CSF-containing space of the subject, the implantable access port device may permit infusion of therapeutic fluid and aspiration of CSF through separate lumens of the brain catheter and through separate fluid paths of the device.

For example, fluid may be aspirated from the CSF-containing space of the subject through the aspiration lumen of the brain catheter and through the aspiration path of the implantable access port device. A lumen of a needle may be placed in communication with the aspiration fluid path of the implantable access port device. The fluid may be aspirated from the CSF-containing space of the subject through lumen of the needle.

In some embodiments, the aspiration path of the implantable access port device and the aspiration lumen of the brain catheter may be used to infuse fluid, such as a fluid comprising a therapeutic agent, into the CSF-containing space of the subject. For example, a lumen of a needle may be placed in communication with the aspiration path of the implantable access port device. The fluid may be infused through the lumen of the needle, through the aspiration path of the implantable access port device, and through the aspiration lumen of the brain catheter to the CSF-containing space of the subject.

A fluid may be infused to the CSF-containing space of the subject by infusing the fluid through the infusion path of the implantable access port device and through the infusion of the brain catheter or through a catheter coupled to an implantable infusion device and the brain catheter without passing through the access port device. The fluid may comprise a therapeutic agent.

By providing separate lumens and fluid paths, infusion and aspiration (or infusion) may be conducted simultaneously. Accordingly, infusion of a first therapeutic fluid does not need to be disrupted to aspirate a sample of CSF or to introduce a second therapeutic fluid to the CSF. Such lack of disruption of infusion of the first therapeutic fluid may provide for improved therapy.

The devices, kits, and systems described herein may extend the space and time a therapeutic agent is available to its brain target improving pharmacokinetics and pharmacodynamics of the therapeutic agent in the brain relative to prior approaches for direct infusion into the central nervous system.

CSF aspirated via the aspiration path may be used for any suitable purpose. For example, aspirated CSF may be used to determine the concentration of a therapeutic agent, which may be introduced via a therapeutic fluid infused through the second fluid path to the CSF. The flow rate or pattern of infused therapeutic may be adjusted based on concentrations of therapeutic agent determined to be present in the aspirated CSF.

The aspirated CSF may be used to determine whether the subject may have an infection due to implantation or use of the implantable cranial medical device and associated devices. If, for example, infectious bacteria, fungi, or viruses are detected in the aspirated CSF, antibiotics, anti-fungal agents, or antiviral agents may be administered to the CSF through, for example the first fluid flow path of the cranial medical device and first lumen of the brain catheter. Concentration or presence of infectious pathogens in subsequently aspirated CSF may be used to determine whether parameters of therapy should be adjusted or whether the cranial medical device and associated devices should be explanted.

The aspirated CSF may be used to detect or diagnose serious bacterial, fungal and viral infections, including meningitis, encephalitis and syphilis. The aspirated CSF may be used to detect or diagnose bleeding around the brain (subarachnoid hemorrhage). The aspirated CSF may be used to detect or diagnose certain cancers involving the brain or spinal cord. The aspirated CSF may be used to detect or diagnose certain inflammatory conditions of the nervous system, such as multiple sclerosis and Guillain-Barre syndrome.

For example, normal CSF white blood cell count is between 0 and 5, and normal CSF red blood cell count is 0. An increase of white blood cells may indicate infection or inflammation, an increase in red blood cell count may indicate bleeding into the cerebrospinal fluid.

The aspirated CSF may be used to determine whether the presence or concentration of a biological marker associated with the disease being treated changes in response to the infused therapeutic fluid. In fact, all diseases of the central nervous system have a CSF profile that is unique to the disease and to the progression of the disease. For ALS, markers may include a change in one or more molecules associated with inflammation. For Alzheimer's disease markers may include beta amyloid and tau. For Huntington's disease, a marker may include the hunitingtin protein. For Parkinson's disease, markers may include tau and leucine-rich repeat kinase 2 (LRRK-2).

The aspirated CSF may be used to determine the presence of biomarkers associated with diagnosis and prognosis. Such biomarkers include microRNA such as miR-146a and miR-134 as biomarkers for epilepsy diagnosis and prognosis. The association of miR-146a and miR-134 and epilepsy is described in, for example, Leontariti, et al., “Circulating miR-146a and miR-134 in predicting epilepsy in patients with focal impaired awareness seizures,” Epilepsia, May 2020 (e-published Apr. 21, 2020); 61(5):959-970, which article is hereby incorporated herein by reference in its entirety to the extent that it does not conflict with the disclosure presented herein.

As another example, a seizure event may cause a change in the CSF profile of an individual. More specifically, immunoglobulin synthesis, elevated lactate, cell count, glucose and total protein concentrations as well as blood-brain barrier dysfunction are frequently observable in the CSF profile following epileptic seizures. Blood cell count may be a marker indicative of seizure disorder. For example, higher blood cell counts may be indicative of more severe disorder and often longer hospital stays.

The infusion parameters or other parameters of therapy may be changed based on the presence of concentration of the biological marker in the aspirated CSF.

The aspirated CSF may be used to determine whether the presence or concentration of a biological marker indicative of toxicity of a therapeutic fluid infused into the CSF (e.g., via the second fluid path and second lumen of the catheter). For example, the presence of antibodies or other inflammatory response to the infused therapeutic agent may be indicative of potential toxicity of the infused therapeutic agent. The presence or increased concentrations of one or more of S100 proteins, neuron specific enolase (NSE), tau, and beta-amyloid may be indicative of toxicity. If a biomarker indicative of toxicity is detected in aspirated CSF, the rate of infusion of therapeutic fluid may be reduced or infusion of the therapeutic fluid may be discontinued.

In some embodiments, intracranial pressure may be determined by placing a pressure monitor in fluid communication with the first fluid path of the implanted cranial medical device. For example, a needle operably coupled to the pressure sensor may be placed in the port to determine intracranial pressure.

In some embodiments, aspirated CSF combined with therapeutic agents and the infused back into the CSF-containing space.

In some embodiments, a dye or radioactive substances (ventriculograpy, cisternography) may be infused into cerebrospinal fluid to facilitate the making of diagnostic images of the fluid's flow within a subject's brain.

Any suitable therapeutic fluid may be infused through the aspiration or infusion fluid path of the implantable access port device and the lumens of the brain catheter. The therapeutic fluid may comprise any suitable therapeutic agent. Preferably, the therapeutic agent is an agent for treating a disease of the brain. Examples of diseases of the brain include any diseases with pathology or dysfunction occurring in any component of the brain (including the cerebral hemispheres, diencephalon, brain stem, and cerebellum) or the spinal cord. Examples include but are not limited to Parkinson's disease, Alzheimer's disease, dementia, Amyotrophic Lateral Sclerosis, Huntington's disease, lysosomal storage diseases, post-traumatic stress disorder, anxiety, depression, brain tumors, autism, autism spectrum disorder, closed head injury, spinal cord injury, stroke, multiple sclerosis, schizophrenia, anxiety, and epilepsy. Preferably, the implantable cranial medical device and associated devices are used to treat a disease of the brain that is resistant to treatment through systemic routes of administration, such as oral, intravenous, intramuscular, and intraperitoneal administration. The implantable cranial medical device and associated devices may also be used if a patient is at serious risk if direct central administration of a therapeutic fluid is not commenced.

The therapeutic fluid may be infused into the CSF or other brain region at any suitable rate. Preferably, flow rate into the brain is limited to 20 milliliters or less per day, such as 10 milliliters or less per day, or 5 milliliters per day or less. For example, the therapeutic fluid may be infused at a metered rate of 4 milliliters per day or less, such as 3 milliliters per day or less, 2 milliliters per day or less, or about 1 milliliter per day.

In embodiments, withdrawal of CSF from the brain is limited 100 milliliters per day. In embodiments, withdrawal of CSF from the brain is limited to 50 milliliters per day. In embodiments, withdrawal of CSF from the brain is limited to 25 milliliters per day. In embodiments, withdrawal of CSF from the brain is limited to 10 milliliters per day. For any given CSF withdrawal event, 7 milliliters or less of CSF is removed from the brain.

The therapeutic fluid may comprise any suitable therapeutic agent. The therapeutic agent selected may depend on the disease being treated. The therapeutic fluid, such as a solution, may contain any suitable concentration of the therapeutic agent. The concentration of the therapeutic agent will depend on the therapeutic agent employed. In some embodiments, the therapeutic fluid is a solution comprising a therapeutic agent at a concentration in a range of from about 10 milligrams per milliliter to about 500 milligrams per milliliter, such as from about 50 milligrams per milliliter to about 450 milligrams per milliliter.

For purposes of illustration, a list of suitable anti-epileptic therapeutic agent that may be included in a therapeutic fluid, such as a solution, includes carbamazepine; tiagabine, levetiracetam; lamotrigine; pregabalin; fenfluramine; gabapentin; phenytoin; topiramate; oxcarbazepine; valproate; valproic acid; zonisamide; perampanel; eslicarbazepine acetate; lacosamide; vigabatrin; rufinamide; fosphenytoin; ethosuximide; phenobarbital; diazepam; lorazepam; clonazepam; clobazam; ezogabine; felbamate; primidone; acetazolamide; brivaracetam; clorazepate; ethotoin; mephenytoin; methsuximide; trimethadione; bumetanide; adenosine; and an adenosine al receptor agonist. In some embodiment, the therapeutic agent is valproic acid or a pharmacologically acceptable salt thereof. For purposes of the present disclosure, reference to a compound includes reference to salts, hydrates, solvates, and polymorphs thereof.

Examples of other therapeutic agents that may be delivered using an implantable infusion device and system as described herein for treating or diagnosing a CNS disease include Edaravone (e.g., Radicava®) for Amyotrophic Lateral Sclerosis (ALS), Valbenazine (e.g., Ingrezza®) for Tardive dyskinesia, Deuterabenazine (e.g., Austedo®) for Huntington's disease, Ocrelizumab (e.g., Ocrevus®) for Multiple sclerosis, Safinamide (e.g., Xadago®) for Parkinson's disease, Nusinersen (e.g., Spinraza®) for Spinal muscular atrophy (SMA), Daclizumab (e.g., Zinbryta®) for Multiple sclerosis, Pivavanserin (e.g., Nuplazid®) for Hallucinations and delusions associated with psychosis, Ariprprazole lauroxil (e.g., Aristada®) for Schizophrenia, Caripazine (e.g., Vraylar®) for Schizophrenia and bipolar disorder, Brexpiprazole (e.g., Rexulti®) for Schizophrenia, Peginterferon beta-la (e.g., Plegridy®) for Multiple sclerosis, Eslicarbazepine acetate (Aptiom®) for Epilepsy associated seizures, Flutemetamol F 18 (e.g., Vizamyl®) Radioactive diagnostic for Alzheimer's disease, Vortioxetine (e.g., Brintellix®) for Major depressive disorder, Dimethyl fumerate (e.g., Tecfidera®) for multiple sclerosis, and Gadoterate megumine (e.g., Dotarem®) for MM-based brain imaging.

Summary of Some Selected Aspects

Various aspects of implantable cranial medical devices, systems and kits including implantable cranial medical devices, methods for implanting implantable cranial medical devices and associated devices, and methods for using implantable cranial medical devices and associated devices are discloses herein. A summary of some of the aspect is provided below.

In a first aspect, a device is configured to be implanted between a scalp and a skull of a subject. The device comprises (a) a housing having a bottom configured to face the skull and a top configured to face the scalp; (b) a first catheter connector configured to couple to a first dual lumen catheter, wherein the first catheter connector extends from the housing and comprises a first lumen and a second lumen; (c) a second catheter connector configured to couple to a single lumen catheter, wherein the second catheter connector extends from the housing; (d) an opening defined by the top of the housing, wherein the opening is configured to be accessed by a needle percutaneously inserted through the scalp when the device is implanted; (e) a first fluid pathway from the first lumen of the first catheter connector to the opening defined by the top of the housing; and (f) a second fluid pathway from the second lumen of the first catheter connector to the second catheter connector.

A second aspect is the device of the first aspect, comprising a self-sealing septum disposed across the opening defined by the top of the housing.

A third aspect is the device of the first or second aspects, comprising a first protective covering extending away from the opening and over the first catheter connector port a distance from 0.5 cm to 5 cm.

As fourth aspect is the device of the third aspect, wherein the first protective covering comprises a bottom surface configured to be placed on the skull, a top surface opposing the bottom surface, and a groove formed in the bottom surface, wherein the groove is configured to receive the first dual lumen catheter.

A fifth aspect is the device of any one of aspects 1 to 4, comprising a second protective covering extending away from the opening and over the second catheter connector port a distance from 0.5 cm to 5 cm.

A sixth aspect is the device of the fifth aspect, wherein the second protective covering comprises a bottom surface configured to be placed on the skull, a top surface opposing the bottom surface, and a groove formed in the bottom surface, wherein the groove is configured to receive the single lumen catheter.

A seventh aspect is the device of any one of aspects 3 to 6, wherein one or both of the first protective covering and the second protective covering are part of the device housing.

An eight aspect is the device of any one of aspects 3 to 6, wherein one or both of the first protective covering and second protective covering are attached to the housing or are configured to attach to the housing.

A ninth aspect is a system comprising: (a) the device of any one of aspects 1-8; (b) the first dual lumen catheter configured to connect to the first catheter connector port; and (c) the single lumen catheter configured to couple to the second catheter connector port.

A tenth aspect is the system of the ninth aspect, wherein the first dual lumen catheter comprises a first opening in communication with the first lumen, and a second opening in communication with the second lumen, and wherein the first and second openings of the dual lumen catheter are configured to be placed in a brain when the first dual lumen catheter is coupled to the first port of the device.

An eleventh aspect is the system of the tenth aspect, wherein the first dual lumen catheter has a length from 55 millimeters to 80 millimeters.

A twelfth aspect is the system the tenth or eleventh aspect, wherein a portion of the first dual lumen catheter that is configured to couple to the first port is resistant to puncture by the needle.

A thirteenth aspect is the system of the twelfth aspect, wherein the portion of the first dual lumen catheter that is resistant to puncture by the needle extends from an end of the single lumen catheter a distance from 0.5 cm to 5 cm.

A fourteenth aspect is the system of the ninth aspect, comprising a dual lumen catheter connector and a second dual lumen catheter having a first lumen and a second lumen, wherein the dual lumen catheter connector is configured to operably coupled the first dual lumen catheter to the second dual lumen catheter such that the first lumen of the first dual lumen catheter is placed in fluid communication with the first lumen of the second dual lumen catheter and such that the second lumen of the first dual lumen catheter is placed in fluid communication with the second lumen of the second dual lumen catheter.

A fifteenth aspect is the system of the fourteenth aspect, wherein the first dual lumen catheter has a length from 0.5 cm to 5 cm.

A sixteenth aspect is the system of the fourteenth or fifteenth aspect, wherein the first dual lumen catheter is resistant to puncture by the needle.

A seventeenth aspect is the system of the fourteenth or the fifteenth aspect, wherein the second dual lumen catheter has a length from 0.5 cm to 5 cm.

An eighteenth aspect is the system of any one of aspects 9 to 17, wherein a portion of the single lumen catheter that is configured to couple to the second port is resistant to puncture by the needle.

A nineteenth aspect is the system of the eighteenth aspect, wherein the portion of the single lumen catheter that is resistant to puncture by the needle extends from an end of the single lumen catheter a distance from 0.5 cm to 5 cm.

A twentieth aspect is the system of any one of aspects 9 to 19, comprising an implantable infusion pump, wherein the implantable infusion pump is configured to connect to the single lumen catheter.

A twenty-first aspect is a system comprising: (a) an access port device configured to be implanted between a scalp and a skull of a subject, the access port device comprising: (i) an opening configured to be accessed by a needle percutaneously inserted through the scalp when the access port device is implanted; (ii) a catheter connector; and (iii) a fluid pathway extending from the catheter connector to the opening; (b) a first catheter comprising a first lumen and a second lumen, wherein the first catheter comprises a first opening in communication with the first lumen and a second opening in communication with the second lumen, wherein the first and second openings are configured to be implanted in a brain of the subject; (c) a second catheter configured to be placed in communication with the first lumen and configured to be connected to the catheter connector of the access port device; (d) a third catheter comprising a fourth lumen configured to be placed in communication with the second lumen of the first catheter and configured to be connected to an implantable infusion pump.

A twenty-second aspect is the system of the twenty-first aspect, further comprising a dual lumen to two single lumen catheter connector configured to couple the first catheter to the second and third catheters, such that the first lumen of the first catheter is in communication with the third lumen of the second catheter and the second lumen of the first catheter is in communication with the fourth lumen of the third catheter.

A twenty-third aspect is a system comprising (a) an access port device configured to be implanted between a scalp and a skull of a subject, the access port device comprising: (i) an opening configured to be accessed by a needle percutaneously inserted through the scalp when the access port device is implanted; (ii) a catheter connector; and (iii) a fluid pathway extending from the catheter connector to the opening; and (b) a catheter comprising a first lumen and a second lumen, wherein the catheter comprises a first opening in communication with the first lumen and a second opening in communication with the second lumen, wherein the first and second openings are configured to be implanted in a brain of the subject, wherein a first portion of the catheter comprising the first lumen is configured to be connected to the catheter connector of the access port device, and wherein a second portion of the catheter comprising the second lumen is configured to be connected to an implantable infusion pump.

A twenty-fourth aspect is a system of any one of aspects 21 to 23, further comprising the implantable infusion device.

All scientific and technical terms used herein have meanings commonly used in the art unless otherwise specified. The definitions provided herein are to facilitate understanding of certain terms used frequently herein and are not meant to limit the scope of the present disclosure.

As used herein, singular forms “a,” “an” and “the” include plural referents unless the context clearly dictates otherwise. As used in this specification and the appended claims, the term “or” is generally employed in its sense including “and/or” unless the content clearly dictates otherwise. The term “and/or” means one or all of the listed elements or a combination of any two or more of the listed elements.

The words “preferred” and “preferably” refer to embodiments of the disclosure that may afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the inventive technology.

Any direction referred to herein, such as “top,” “bottom,” “side,” “upper,” “lower,” and other directions or orientations are described herein for clarity and brevity but are not intended to be limiting of an actual device or system. Devices and systems described herein may be used in a number of directions and orientations.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that any particular order be inferred. Any recited single or multiple feature or aspect in any one claim can be combined or permuted with any other recited feature or aspect in any other claim or claims.

The headings used herein are for organizational purposes only and are not meant to be used to limit the scope of the description. As used throughout this application, the word “may” is used in a permissive sense (i.e., meaning having the potential to), rather than the mandatory sense (i.e., meaning must).

The words “include,” “including,” and “includes” indicate open-ended relationships and therefore mean including, but not limited to. Similarly, the words “have,” “having,” and “has” also indicated open-ended relationships, and thus mean having, but not limited to. Similarly, the terms “comprise” and “comprising” indicate open-ended relationships, and thus mean comprising, but not limited to. The terms “consisting essentially of” and “consisting of” are subsumed within the term “comprising.” For example, a catheter comprising tubing may be a catheter consisting of tubing. The term “consisting essentially of” means a recited list of one or more items belonging to an article, kit, system, or method and other non-listed items that do not materially affect the properties of the article, kit, system, or method.

The terms “first,” “second,” “third,” and so forth as used herein are used as labels for nouns that they precede, and do not imply any type of ordering (e.g., spatial, temporal, logical, etc.) unless such an ordering is otherwise explicitly indicated. For example, a “second” feature does not require that a “first” feature be implemented prior to the “second” feature, unless otherwise specified.

Various components may be described as “configured to” perform a task or tasks. In such contexts, “configured to” is a broad recitation generally meaning “having structure that” performs the task or tasks during operation. As such, the component can be configured to perform the task even when the component is not currently performing that task (e.g., a catheter connector may be configured to place a lumen of a catheter in fluid communication with a fluid path, even when the catheter is not connected to the catheter connector).

Various components may be described as performing a task or tasks, for convenience in the description. Such descriptions should be interpreted as including the phrase “configured to.” Reciting a component that is configured to perform one or more tasks is expressly intended not to invoke 35 U.S.C. § 112 paragraph (f), interpretation for that component

As used herein, a catheter that is “resistant to puncture” is a catheter that does not puncture when contacted with a 22 gauge Huber (non-coring) needle under typical manual insertion force applied when a medical professional, using due care, inserts a needle through the scalp to access a septum-covered opening of a port positioned between the skull and the scalp.

It will be apparent to those skilled in the art that various modifications and variations can be made to the present inventive technology without departing from the spirit and scope of the disclosure. Since modifications, combinations, sub-combinations and variations of the disclosed embodiments incorporating the spirit and substance of the inventive technology may occur to persons skilled in the art, the inventive technology should be construed to include everything within the scope of the appended claims and their equivalents.

What is claimed is:

1. A device configured to be implanted between a scalp and a skull of a subject, the device comprising:
a housing having a bottom configured to face the skull and a top configured to face the scalp;
a first catheter connector configured to couple to a first dual lumen catheter, wherein the first catheter connector extends from the housing and comprises a first lumen and a second lumen;
a second catheter connector configured to couple to a single lumen catheter, wherein the second catheter connector extends from the housing;
an opening defined by the top of the housing, wherein the opening is configured to be accessed by a needle percutaneously inserted through the scalp when the device is implanted;
a first fluid pathway from the first lumen of the first catheter connector to the opening defined by the top of the housing; and
a second fluid pathway from the second lumen of the first connector to the second catheter connector.

2. The device of claim 1, comprising a self-sealing septum disposed across the opening defined by the top of the housing.

3. The device of claim 1, comprising a first protective covering extending away from the opening and over the first catheter connection port a distance from 0.5 cm to 5 cm.

4. The device of claim 3, wherein the first protective covering comprises a bottom surface configured to be placed on the skull, a top surface opposing the bottom surface, and a groove formed in the bottom surface, wherein the groove is configured to receive the first dual lumen catheter.

5. The device of claim 1, comprising a second protective covering extending away from the opening and over the second catheter connection port a distance from 0.5 cm to 5 cm.

6. The device of claim 5, wherein the second protective covering comprises a bottom surface configured to be placed on the skull, a top surface opposing the bottom surface, and a groove formed in the bottom surface, wherein the groove is configured to receive the single lumen catheter.

7. The device of claim 3, wherein one or both of the first protective covering and the second protective covering are part of the device housing.

8. The device of claim 3, wherein one or both of the first protective covering and the second protective covering are attached to the housing or are configured to attach to the housing.

9. A system comprising:
the device of claim 1;
the first dual lumen catheter configured to connect to the first catheter connection port; and
the single lumen catheter configured to couple to the second catheter connection port.

10. The system of claim 9, wherein the first dual lumen catheter comprises a first opening in communication with the first lumen, and a second opening in communication with the second lumen, and wherein the first and second openings of the dual lumen catheter are configured to be placed in a brain when the first dual lumen catheter is coupled to the first port of the device.

11. The system of claim 10, wherein the first dual lumen catheter has a length from 55 millimeters to 80 millimeters.

12. The system of claim 10, wherein a portion of the first dual lumen catheter that is configured to couple to the first port is resistant to puncture by the needle.

13. The system of claim 12, wherein the portion of the first dual lumen catheter that is resistant to puncture by the needle extends from an end of the single lumen catheter a distance from 0.5 cm to 5 cm.

14. The system of claim 9, comprising a dual lumen catheter connector and a second dual lumen catheter having a first lumen and a second lumen, wherein the dual lumen catheter connector is configured to operably coupled the first dual lumen catheter to the second dual lumen catheter such that the first lumen of the first dual lumen catheter is placed in fluid communication with the first lumen of the second dual lumen catheter and such that the second lumen of the first dual lumen catheter is placed in fluid communication with the second lumen of the second dual lumen catheter.

15. The system of claim 14, wherein the first dual lumen catheter has a length from 0.5 cm to 5 cm.

16. The system of claim 14, wherein the first dual lumen catheter is resistant to puncture by the needle.

17. The system of claim 14, wherein the second dual lumen catheter has a length from 0.5 cm to 5 cm.

18. The system of claim 9, wherein a portion of the single lumen catheter that is configured to couple to the second port is resistant to puncture by the needle.

19. The system of claim 18, wherein the portion of the single lumen catheter that is resistant to puncture by the needle extends from an end of the single lumen catheter a distance from 0.5 cm to 5 cm.

20. The system of claim 9, comprising an implantable infusion pump, wherein the implantable infusion pump is configured to connect to the single lumen catheter.

21. A system comprising:
an access port device configured to be implanted between a scalp and a skull of a subject, the access port device comprising:
an opening configured to be accessed by a needle percutaneously inserted through the scalp when the access port device is implanted;
a catheter connector; and
a fluid pathway extending from the catheter connector to the opening;
a first catheter comprising a first lumen and a second lumen, wherein the first catheter comprises a first opening in communication with the first lumen and a second opening in communication with the second lumen, wherein the first and second openings are configured to be implanted in a brain of the subject;
a second catheter configured to be placed in communication with the first lumen and configured to be connected to the catheter connector of the access port device;
a third catheter comprising a fourth lumen configured to be placed in communication with the second lumen of the first catheter and configured to be connected to an implantable infusion pump.

22. The system of claim 21, further comprising a dual lumen to two single lumen catheter connector configured to couple the first catheter to the second and third catheters, such that the first lumen of the first catheter is in communication with the third lumen of the second catheter and the second lumen of the first catheter is in communication with the fourth lumen of the third catheter.

23. The system of claim 21, further comprising the implantable infusion device.

24. A system comprising:
an access port device configured to be implanted between a scalp and a skull of a subject, the access port device comprising:
an opening configured to be accessed by a needle percutaneously inserted through the scalp when the access port device is implanted;
a catheter connector; and
a fluid pathway extending from the catheter connector to the opening; and
a catheter comprising a first lumen and a second lumen, wherein the catheter comprises a first opening in communication with the first lumen and a second opening in communication with the second lumen, wherein the first and second openings are configured to be implanted in a brain of the subject,
wherein a first portion of the catheter comprising the first lumen is configured to be connected to the catheter connector of the access port device,
wherein a second portion of the catheter comprising the second lumen is configured to be connected to an implantable infusion pump.

25. The system of claim 24, further comprising the implantable infusion device.
